(19) United States
(12) Patent Application Publication
(10) Pub. No.: US 2024/0139358 A1
(43) Pub. Date: May 2, 2024

(54) ULTRAVIOLET STERILIZATION LAMP STRUCTURE AND ULTRAVIOLET STERILIZATION LAMP

(71) Applicant: SHENZHEN YITOA INTELLIGENT HEALTH TECHNOLOGY CO., LTD, SHENZHEN CITY (CN)

(72) Inventors: SHUJIE WANG, SHENZHEN CITY (CN); JUNHUANG ZHUANG, SHENZHEN CITY (CN); ZHIHONG ZHENG, SHENZHEN CITY (CN)

(21) Appl. No.: 18/075,492

(22) Filed: Dec. 6, 2022

(30) Foreign Application Priority Data
Oct. 28, 2022 (CN) 202222871888.9

(51) Int. Cl. A61L 2/10; A61L 2/26; F21V 5/00; F21V 5/04

(52) U.S. Cl. CPC A61L 2/10; A61L 2/26; F21V 5/008; F21V 5/04; F21Y 2107/40

(57) ABSTRACT

An ultraviolet sterilization lamp structure relates to the technical field of disinfection and sterilization, comprising: a lamp board; an ultraviolet lamp connected to the lamp board, a light source convex lens arranged on a positive side of the ultraviolet lamp, and a strip-shaped convex lens laterally arranged on an outer side of the light source convex lens. With the above technical solution, the ultraviolet light is optically zoomed twice through the light source convex lens and the strip-shaped convex lens to achieve an effect of vertical concentration and horizontal diffusion, ensuring that the horizontal angle of the irradiation is large and the vertical angle is small, avoiding the exposure to the personnel activity area, so that the personnel do not have to leave the room when sterilizing, thereby embodying the coexistence of man and machine and improving the immediacy and practicality of ultraviolet air sterilizing.

### FIELD OF THE DISCLOSURE

The present disclosure relates to the technical field of disinfection and sterilization, in particular, to an ultraviolet sterilization lamp structure and an ultraviolet sterilization lamp.

### BACKGROUND OF THE DISCLOSURE

The transmission route of highly contagious viruses is usually airborne. For most of the indoors that cannot be ventilated, air conditioners, fresh air systems, ceiling fans, or other equipment are relied upon for convection. Such devices have the characteristic of allowing airflow through the upper area of the space, and the upper area of the indoor space is the necessary range for the circulating air. Therefore, ultraviolet irradiation and disinfection of the air in the upper indoor space can greatly reduce the risk of indoor cross-infection. However, most of the ultraviolet lamps currently used for irradiation and sterilization have a large scattering range, which is easy to irradiate the area for human activity. Further, it is easy to generate ozone, which increases the risk of use. Therefore, personnel must avoid and cannot coexist with it during disinfection, which reduces the practicality of indoor air disinfection.

### SUMMARY OF THE DISCLOSURE

The purpose of the present disclosure is to provide an ultraviolet sterilization lamp structure in view of the defects and deficiencies of the prior art, which has the advantages of being able to irradiate in a directional range, embody the coexistence of man and machine and improve the immediacy and practicability of sterilizing ultraviolet air.

For achieve the above purpose, the present disclose provides an ultraviolet sterilization lamp structure, comprising: a lamp board; an ultraviolet lamp connected to the lamp board; a light source convex lens arranged on a positive side of the ultraviolet lamp; and a strip-shaped convex lens laterally arranged on an outer side of the light source convex lens.

In certain embodiments, a symmetry axis of the strip-shaped convex lens is aligned with the ultraviolet lamp.

In certain embodiments, multiple ultraviolet lamps are arranged laterally on the lamp board.

The present disclosure further provides an ultraviolet sterilization lamp, comprising: a lamp body; at least one lamp board disposed on the lamp body; at least one ultraviolet lamp disposed on the lamp board and connected to the at least one lamp board; a light source convex lens disposed on the ultraviolet lamp; and a strip-shaped convex lens disposed on the lamp body; wherein a symmetry axis of the strip-shaped convex lens is aligned with the at least one ultraviolet lamp.

In certain embodiments, the lamp body is octagonal, and a number of the lamp boards is eight.

In certain embodiments, the lamp board is provided with two rows of the ultraviolet lamps, and each of the two rows of the ultraviolet lamps corresponds to one of the strip-shaped convex lenses.

In certain embodiments, a number of the ultraviolet lamps is five per row.

In certain embodiments, the lamp body is provided with a top cover and a bottom cover.

In certain embodiments, the lamp body comprises a center body and an extended assembly edge provided on an edge of the center body, and the strip-shaped convex lens is arranged between the extended assembly edges.

In certain embodiments, the lamp body is triangular, and a number of the lamp boards is three.

After adopting the above-mentioned technical solution, the beneficial effects of the present disclosure are as follows.

1. The ultraviolet light passes through the light source convex lens and the strip-shaped convex lens to perform optical zoom twice to achieve an effect of vertical concentration and horizontal diffusion, thereby forming a wide and flat light spot. Therefore, the horizontal angle of the irradiation can be ensured to be large while the vertical angle is small, which can ensure the range of ultraviolet irradiation, but also ensure that the ultraviolet light stays in the upper area to avoid exposure to the area for human activity, so that the personnel do not have to leave the room when sterilizing, thereby embodying the coexistence of man and machine and improving the immediacy and practicality of ultraviolet air sterilizing.

2. Multiple ultraviolet lamps are arranged around the lamp body, and the strip-shaped convex lens is used to increase the horizontal irradiation angle, so that the ultraviolet lights can more easily cover the surroundings of the lamp, achieve 360-degree irradiation, increase the sterilizing area of a single lamp, and improve the use efficiency.

### BRIEF DESCRIPTION OF THE DRAWINGS

In order to more clearly illustrate the embodiments of the present disclosure or the technical solutions in the prior art, the following briefly introduces the accompanying drawings that need to be used in the description of the embodiments or the prior art. Obviously, the accompanying drawings in the following description are just some embodiments of the present disclosure, and for those of ordinary skill in the art, other drawings can also be obtained from these drawings without any creative effort.

FIG. 1 is a schematic diagram of a structure of the present disclosure.

FIG. 2 is a schematic exploded diagram of the structure of the pre sent disclosure.

FIG. 3 is another schematic exploded diagram of the structure of the present disclosure.

FIG. 4 is a schematic diagram of an ultraviolet lamp and a convex lens of the present disclosure.

FIG. 5A is a schematic diagram of a top view effect of the pre sent disclosure.

FIG. 5B is a schematic diagram of a side view effect of the pre sent disclosure.

### REFERENCE NUMERAL

1. lamp board; 2. ultraviolet lamp; 3. light source convex lens; 4. strip-shaped convex lens; 5. lamp body; 51. center body; 52. extended assembly edge; 61. top cover; 62. bottom cover.

### DETAILED DESCRIPTION OF THE EXEMPLARY EMBODIMENTS

The present disclosure will be described in further detail below in conjunction with the accompanying drawings.

This specific embodiment is only an explanation of the present disclosure, and it is not a limitation of the present disclosure. Those skilled in the art can make modifications to this embodiment without creative contribution as needed after reading this specification, but as long as they are within the scope of the claims of the present disclosure, they are protected by the patent law.

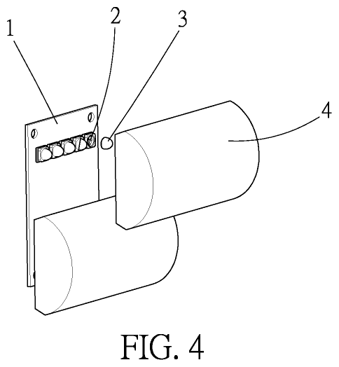

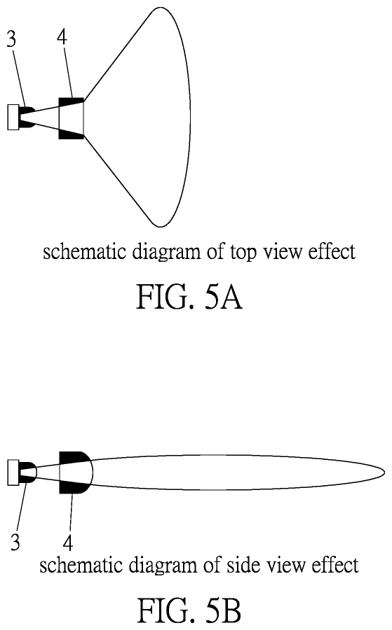

The present embodiment relates to an ultraviolet sterilization lamp structure, which mainly includes a lamp board 1, an ultraviolet lamp 2 connected to the lamp board 1, a light source convex lens 3 arranged on a positive side of the ultraviolet lamp 2, and a strip-shaped convex lens 4 laterally arranged on an outer side of the light source convex lens 3. The light source convex lens 3 and the ultraviolet lamp 2 are integrally formed, and the strip convex lens 4 is a horizontal strip convex lens. As shown in FIG. 4, FIG. 5A and FIG. 5B, the ultraviolet light passes through the two lenses for secondary optical zoom to form a wide and flat spot. Such kind of light spot has the characteristics of large horizontal irradiation area and small vertical irradiation angle, which can ensure that the range of ultraviolet irradiation only stays in the upper area and does not enter the area for human activity. The traditional ultraviolet light tube emits light in a comprehensive radiation method. However, if the reflection method is used to adjust the scattering, the light loss becomes larger, and the reflective optical design is still not easy to prevent the light from entering the area for human activity, so the personnel still need to avoid. The ultraviolet light through the secondary zoom can avoid the vertical irradiation range from being too large. By cooperating with the natural convection of the air or the active convection of HVAC, fresh air and other equipment, during the circulation process, the indoor air can be sterilized by the ultraviolet radiation in the upper area, so as to ensure the overall indoor air can be fully sterilized.

Multiple ultraviolet lamps 2 may be arranged on the lamp board 1, and the multiple ultraviolet lamps 2 are arranged horizontally at the same height of the lamp board 1. For example, five ultraviolet lamps 2 are arranged on the lamp board 1 to improve the efficiency of ultraviolet irradiation, and make full use of the long length of the strip-shaped convex lens 4 to increase the horizontal irradiation area.

In addition, in such ultraviolet sterilization lamp structure, the strip-shaped convex lens 4 is preferably placed horizontally and flushes with the ultraviolet lamp 2, that is, a symmetry axis of the strip-shaped convex lens 4 is aligned with the ultraviolet lamp 2. In the case of a single ultraviolet lamp 2, it is preferably to make the vertical axis of symmetry and the horizontal axis of symmetry pass through a center of the ultraviolet lamp 2. When of the multiple ultraviolet lamps 2 are arranged in a horizontal direction, then the horizontal axis of symmetry of the ultraviolet lamps 2 passes through this row of ultraviolet lamps 2, making the vertical axis of symmetry pass through a center of the ultraviolet lamp 2 that is in the middle. When the ultraviolet light is zoomed, the angles of the vertical illumination change the same.

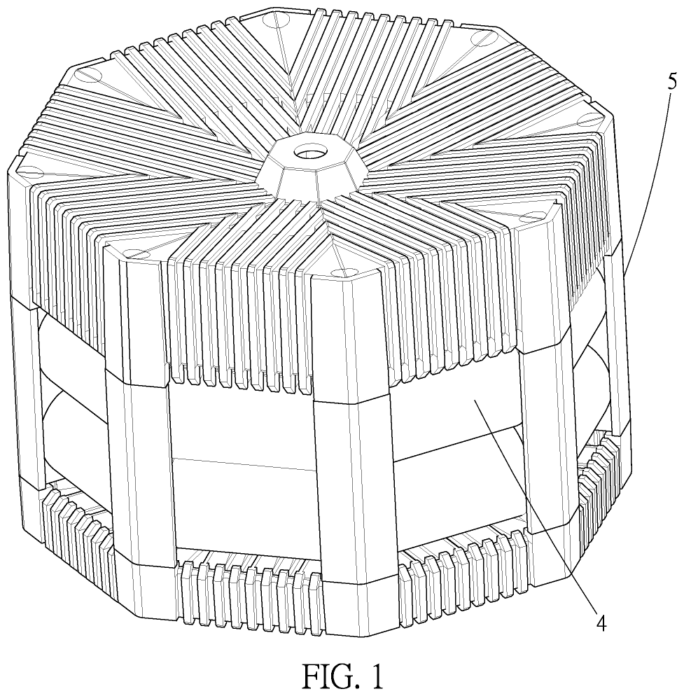

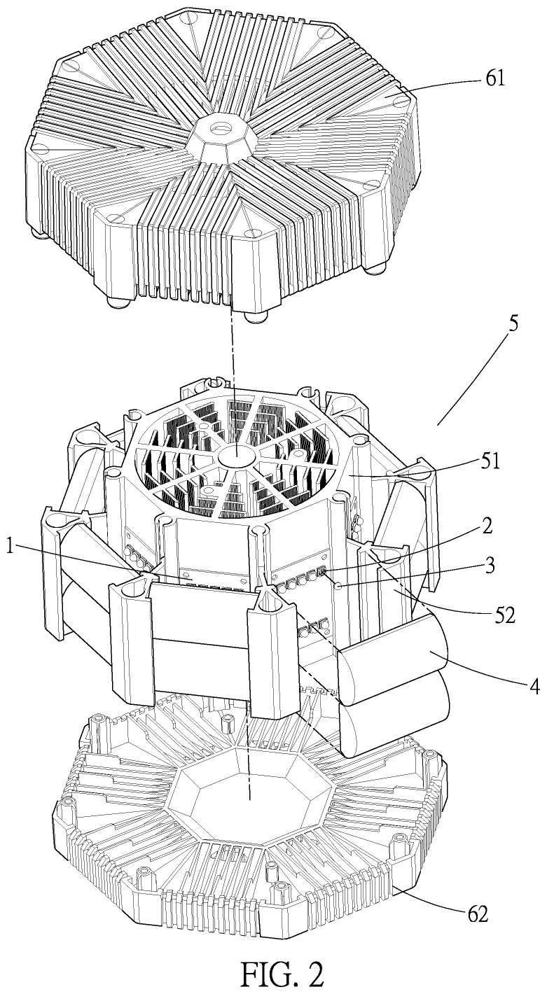

As shown in FIG. 1 and FIG. 2, the present disclosure further provides an ultraviolet sterilization lamp, which mainly includes a lamp body 5, a lamp board 1 disposed on the lamp body 5, an ultraviolet lamp 2 disposed on the lamp board 1 and connected to the lamp board 1, a light source convex lens 3 disposed on the ultraviolet lamp 2, and a strip-shaped convex lens 4 disposed on the lamp body 5. A symmetry axis of the strip-shaped convex lens 4 is aligned with the ultraviolet lamp 2, so that the angle of the vertical illumination changes the same when the ultraviolet lamp is zoomed. By using the combination of LED light source and refracting lens optics, a directional and limited range of irradiation is designed, so that the irradiation range of the ultraviolet light is wide and flat. While irradiating and sterilizing the air, the area for human activity is not irradiated. While sterilizing the air in the upper area, it also protects the safety of the personnel, which is convenient to set up in the upper area for continuous sterilizing. It greatly reduces the risk of using ultraviolet lights, and people do not have to leave the room when sterilizing, so as to achieve the purpose of coexistence of humans and machines, and improve the immediacy and practicability of ultraviolet air disinfection.

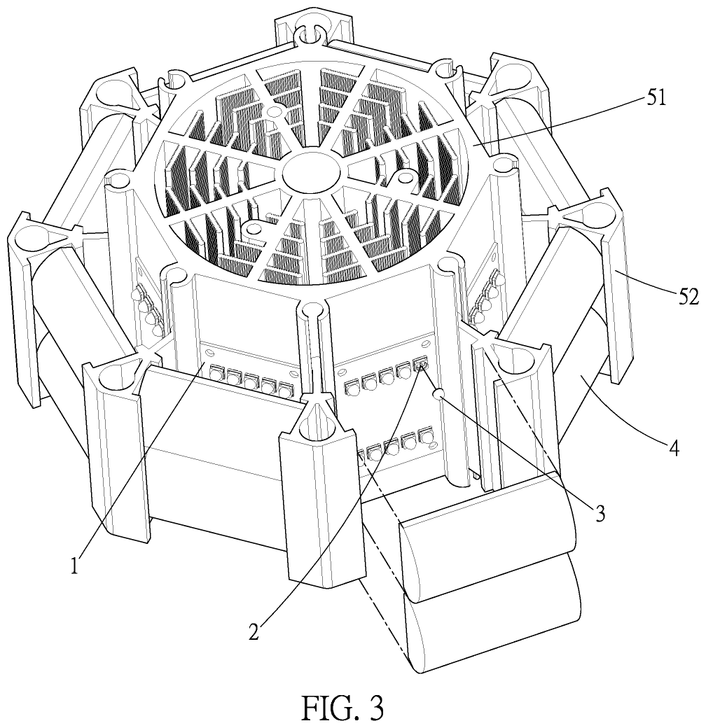

In this embodiment, as shown in FIG. 1 and FIG. 3, the lamp body 5 presents an octagon as a whole, and the number of the lamp boards 1 is eight, which are respectively arranged on the eight sides of the lamp body 5. The multi-faceted lamps can achieve multi-faceted lighting, and in cooperation with the wide and flat irradiation range, the effect of horizontal 360-degree irradiation can be easily achieved, so that the ultraviolet light covers the space around the lamp. In another embodiment, the lamp body 5 presents a triangle as a whole, and the number of the lamp boards 1 is three, which can also achieve horizontal 360-degree light emission, and the same is true for other polygons.

In the lamp of the present embodiment, each lamp board 1 is provided with two rows of ultraviolet lamps 2. As shown in FIG. 2 and FIG. 3, each row of ultraviolet lamps 2 corresponds to a strip-shaped convex lens 4 on the lamp body 5. Specifically, the number of ultraviolet lamps 2 in each row is five, which is suitable for the size of the octagonal lamps. While improving the efficiency of ultraviolet irradiation, the characteristics of the long length of a single strip-shaped convex lens 4 are fully utilized, the lateral irradiation area is increased, and the overall 360-degree irradiation effect is more easily achieved.

The lamp body 5 specifically includes a center body 51 and an extended assembly edge 52 arranged on an edge of the center body 51. As shown in FIG. 3, the strip-shaped convex lens 4 is assembled between the extended assembly edges 52, and the extended assembly edge 52 is assembled to the central body 51 by snapping into a mounting groove on the edge of the central body 51. The lamp body 5 is provided with a top cover 61 and a bottom cover 62, which enhances a stability of the structure and prevents the ultraviolet light emitted by the ultraviolet lamp 2 from diffusing vertically after passing through the light source convex lens 3, thereby further ensuring the safety of the ultraviolet lamp.

The working principle of the present disclosure is as follows. The light source convex lens 3 and the strip-shaped convex lens 4 are arranged outside the ultraviolet lamp 2, and the light passes through the light source convex lens 3 and the strip-shaped convex lens 4 to perform optical zoom twice, so as to embody the effect of the vertical concentration and horizontal diffusion. By using the combination of LED light source and refracting lens optics, a directional and limited range of irradiation is designed to form a wide and flat spot, ensuring that the horizontal angle of the irradiation is large and the vertical angle is small, which can not only ensure the irradiation range of ultraviolet sterilization, but also ensure ultraviolet lights stay in the upper area. While irradiating the sterilizing air in the upper area, it will not irradiate the area for human activity, so as to protect the safety of the personnel, and it is convenient to set it in the upper area for continuous sterilizing. It greatly reduces the risk of using ultraviolet lights, and people do not have to leave the room when disinfecting, so as to achieve the purpose of coexistence of humans and machines, and improve the immediacy and practicability of ultraviolet air disinfection.

The above is only used to illustrate the technical solution of the present disclosure and not to limit it. Other modifications or equivalent replacements made by those of ordinary skill in the art to the technical solution of the present disclosure are not departing from the spirit and scope of the technical solution of the present disclosure. It should be included in the scope of the claims of the present disclosure.

What is claimed is:

1. An ultraviolet sterilization lamp structure, comprising:
a lamp board (1);
at least one ultraviolet lamp (2) connected to the lamp board (1);
a light source convex lens (3) arranged on a positive side of the ultraviolet lamp (2); and
a strip-shaped convex lens (4) laterally arranged on an outer side of the light source convex lens (3).

2. The ultraviolet sterilization lamp structure according to claim 1, wherein a symmetry axis of the strip-shaped convex lens (4) is aligned with the ultraviolet lamp (2).

3. The ultraviolet sterilization lamp structure according to claim 1, wherein multiple ultraviolet lamps (2) are arranged laterally on the lamp board (1).

4. An ultraviolet sterilization lamp, comprising:
at least one lamp body (5);
at least one lamp board (1) disposed on the at least one lamp body (5);
at least one ultraviolet lamp (2) disposed on the lamp board (1) and connected to the at least one lamp board (1);
a light source convex lens (3) disposed on the at least one ultraviolet lamp (2); and
a strip-shaped convex lens (4) disposed on the at least one lamp body (5);
wherein a symmetry axis of the strip-shaped convex lens (4) is aligned with the at least one ultraviolet lamp (2).

5. The ultraviolet sterilization lamp according to claim 4, wherein the lamp body (5) is octagonal, and a number of the lamp boards (1) is eight.

6. The ultraviolet sterilization lamp according to claim 4, wherein the lamp board (1) is provided with two rows of the ultraviolet lamps (2), and each of the two rows of the ultraviolet lamps (2) corresponds to one of the strip-shaped convex lenses (4).

7. The ultraviolet sterilization lamp according to claim 6, wherein a number of the ultraviolet lamps (2) is five per row.

8. The ultraviolet sterilization lamp according to claim 4, wherein the lamp body (5) is provided with a top cover (61) and a bottom cover (62).

9. The ultraviolet sterilization lamp according to claim 4, wherein the lamp body (5) comprises a center body (51) and an extended assembly edge (52) provided on an edge of the center body (51), and the strip-shaped convex lens (4) is arranged between the extended assembly edges (52).

10. The ultraviolet sterilization lamp according to claim 4, wherein the lamp body (5) is triangular, and a number of the lamp boards (1) is three.
